(19) United States
(12) Patent Application Publication
(10) Pub. No.: US 2026/0171657 A1
(43) Pub. Date: Jun. 18, 2026

(54) WEARABLE DEVICE INCLUDING ELECTRODE FOR DETECTING BIOMETRIC INFORMATION

(71) Applicant: SAMSUNG ELECTRONICS CO., LTD., Suwon-si (KR)

(72) Inventors: Chungsoon PARK, Suwon-si (KR); Jeongho AHN, Suwon-si (KR); Subin SHIN, Suwon-si (KR); Bareum PARK, Suwon-si (KR)

(73) Assignee: SAMSUNG ELECTRONICS CO., LTD., Suwon-si (KR)

(21) Appl. No.: 19/534,015

(22) Filed: Feb. 9, 2026

Related U.S. Application Data
(63) Continuation of application No. PCT/KR2024/009412, filed on Jul. 3, 2024.

(30) Foreign Application Priority Data
Aug. 30, 2023 (KR) 10-2023-0114673
Sep. 22, 2023 (KR) 10-2023-0127234

(51) Int. Cl. H01Q 1/27; A61B 5/00; A61B 5/318; G04G 21/02; G04G 21/04; H01Q 1/50

(52) U.S. Cl. CPC H01Q 1/273; A61B 5/0002; A61B 5/318; A61B 5/681; G04G 21/025; G04G 21/04; H01Q 1/50

(57) ABSTRACT

There is provided a wearable device including a frame, including a conductive portion used as an antenna, a cover coupled to the frame and including an electrode to detect an electrical signal from body when worn, a wireless communication circuit to transmit or receive a radio frequency signal by the antenna, a processor to obtain biometric information of a user by the electrical signal, and a switch circuit connected to the ground of the antenna. The electrode may be connected to the processor through a first electrical path to selectively pass the electrical signal, and may be connected to the switch circuit through a second electrical path to selectively pass the RF signal. The processor may be configured to control the switch circuit such that the electrode is selectively connected to the ground of the antenna.

### CROSS-REFERENCE TO RELATED APPLICATIONS

This application is a continuation of International Application No. PCT/KR 2024/009412, filed on Jul. 3, 2024, which is based on and claims priority to Korean Patent Application No. 10-2023-0114673, filed on Aug. 30, 2023, in the Ministry of Intellectual Property of the Republic of Korea, and to Korean Patent Application No. 10-2023-0127234, filed on Sep. 22, 2023, in the Ministry of Intellectual Property of the Republic of Korea, the disclosures of which are incorporated by reference herein in their entireties.

### BACKGROUND

### 1. Field

The following descriptions relate to a wearable device including an electrode for detecting biometric information.

### 2. Description of Related Art

Various portable communication devices such as a smart phone, a tablet, and a wearable device are being developed. Among them, a smart watch is gaining huge popularity as it provides a health care function using various biometric sensors.

Meanwhile, according to development of communication technology, a communication frequency required for these devices is increasing. Accordingly, design and implementation of an antenna supporting various radio frequencies are required.

The above-described information may be provided as a related art for the purpose of helping understanding of the present disclosure. No argument, admission, or decision is made as to whether any of the above description may be applied as or is a prior art related to the present disclosure.

### SUMMARY

According to an embodiment, a wearable device may comprise a frame including a conductive portion used as an antenna, a cover coupled to the frame and including an electrode configured to detect an electrical signal from a human body when wearing the wearable device, a wireless communication circuit configured to transmit or receive a radio frequency (RF) signal using the antenna, a processor configured to obtain biometric information of a user by using the electrical signal, and a switch circuit connected to a ground of the antenna. The electrode may be coupled to the processor through a first electrical path comprising a first circuit configured to selectively pass the electrical signal, and coupled to the switch circuit through a second electrical path comprising a second circuit configured to selectively pass the RF signal. The processor may be configured to control the switch circuit such that the electrode is selectively connected to the ground of the antenna.

According to an embodiment, an electronic watch may comprise a housing including a first cover, a second cover, and a metal frame disposed between the first cover and the second cover and used as an antenna for a radio frequency (RF) signal. The second cover may include a first electrode and a second electrode that are configured to detect an electrical signal. The electronic watch may comprise at least one first circuit configured to selectively pass the electrical signal, at least one second circuit configured to selectively pass the RF signal, a sensor circuit coupled to the first electrode and the second electrode through the at least one first circuit and configured to process the detected electrical signal, a switch circuit coupled to the first electrode and the second electrode through the at least one second circuit, and coupled to a ground of the antenna, and at least one processor operatively connected to the metal frame, the sensor circuit, and the switch circuit. The at least one processor may be configured to transmit or receive the RF signal using the antenna. The at least one processor may be configured to obtain biometric information of a user using the electrical signal provided from the sensor circuit. The at least one processor may be configured to control the switch circuit such that at least one of the first electrode and the second electrode is selectively connected to the ground of the antenna.

There is provided a wearable device including: a frame including a conductive portion that is an antenna; a cover coupled to the frame and including an electrode configured to detect an electrical signal from a body of a user when the user is wearing the wearable device; a wireless communication circuit configured to any of transmit and receive a radio frequency (RF) signal from the antenna; a processor configured to obtain biometric information of the user by using the electrical signal; and a switch circuit connected to a ground of the antenna, and wherein the electrode is: coupled to the processor through a first electrical path including a first circuit configured to selectively pass the electrical signal; and coupled to the switch circuit through a second electrical path including a second circuit configured to selectively pass the RF signal, and wherein the processor is further configured to control the switch circuit to selectively connect the electrode to the ground of the antenna.

The first electrical path and the second electrical path may include a common path connected to the electrode.

The electrical signal may be a first electrical signal, the electrode may be a first electrode, the cover includes a second electrode configured to detect a second electrical signal, and the second electrode may be: coupled to the processor through a third electrical path including a third circuit configured to selectively pass the second electrical signal; and coupled to the switch circuit through a fourth electrical path including a fourth circuit configured to selectively pass the RF signal.

The processor may be further configured to: obtain the biometric information of the user based on the first electrical signal and the second electrical signal; and control the switch circuit to selectively connect at least one of the first electrode and the second electrode to the ground of the antenna.

The first electrical path and the second electrical path may include a first common path connected to the first electrode, and the third electrical path and the fourth electrical path may include a second common path connected to the second electrode.

The wearable device may further include a first printed circuit board on which the processor and the switch circuit are disposed; a second printed circuit board, positioned between the first printed circuit board and the cover; a flexible printed circuit board connecting the second printed circuit board to the first printed circuit board; and an optical sensor disposed on the second printed circuit board and facing towards the cover.

The second printed circuit board may include a first connection portion electrically connected to the first electrode and a second connection portion electrically connected to the second electrode.

The wearable device may further include a first conductive adhesive member electrically connecting the first connection portion and the first electrode; and a second conductive adhesive member electrically connecting the second connection portion and the first electrode.

The first circuit may be at least partly provided on any of the first printed circuit board and the second printed circuit board, the second circuit may be at least partly provided on any of the first printed circuit board and the second printed circuit board, the third circuit may be at least partly provided on any of the first printed circuit board and the second printed circuit board, and the fourth circuit may be at least partly provided on any of the first printed circuit board and the second printed circuit board.

The wearable device may further include a sensor circuit configured to process the first electrical signal and the second electrical signal, and the sensor circuit disposed on the second printed circuit board.

The processor may include at least one sensor circuit configured to process the first electrical signal and the second electrical signal.

The wearable device may further include a conductive member positioned between the first printed circuit board and the second printed circuit board, and the conductive member may be electrically connected to the switch circuit and the ground of the antenna.

The switch circuit may include: a first pin to which the first electrode may be connected; a second pin to which the conductive member may be connected; and a third pin connected to the ground of the antenna, and the switch circuit may be configured to selectively connect the first pin to at least one of the second pin and the third pin.

The switch circuit may include a fourth pin connected to the ground of the antenna through at least one lumped element, and the switch circuit may be configured to selectively connect the first pin to at least one of the second pin, the third pin, and the fourth pin.

The third pin may be connected to the ground of the antenna through at least one other lumped element.

The first circuit may include any of a resistor and an inductor, and the second circuit may include a capacitor.

The biometric information may include an electrocardiogram.

There is provided an electronic watch including: a housing including a first cover, a second cover, and a metal frame disposed between the first cover and the second cover, the metal frame is at least partly an antenna configured to any of receive and transmit a radio frequency (RF) signal, and the second cover includes a first electrode and a second electrode that are configured to detect an electrical signal; at least one first circuit configured to selectively pass the electrical signal; at least one second circuit configured to selectively pass the RF signal; a sensor circuit coupled to the first electrode and the second electrode through the at least one first circuit and configured to process the electrical signal; a switch circuit coupled to the first electrode and the second electrode through the at least one second circuit, and connected to a ground of the antenna; and at least one processor operatively connected to the metal frame, the sensor circuit, and the switch circuit, and the at least one processor is configured to: any of transmit and receive the RF signal from the antenna, obtain biometric information, of a user, based on the electrical signal provided from the sensor circuit; and control the switch circuit to selectively connect at least one of the first electrode and the second electrode to the ground of the antenna.

The electronic watch may include a conductive member disposed within the housing and electrically connected to the switch circuit, and the at least one processor may be further configured to control the switch circuit to selectively connect the at least one of the first electrode and the second electrode to the conductive member.

The at least one first circuit may include a first element and a second element, the at least one second circuit may include a third element and a fourth element, the first element and the third element may be connected in parallel to the first electrode, and the second element and the fourth element may be connected in parallel to the second electrode.

### BRIEF DESCRIPTION OF THE DRAWINGS

The above and other aspects, features, and advantages of specific embodiments of the present disclosure will be more apparent from the following description with reference to the accompanying drawings, in which:

FIG. 1 is a front perspective view of an exemplary electronic device according to one or more embodiments;

FIG. 2 is a rear perspective view of an exemplary electronic device according to one or more embodiments;

FIG. 3 is an exploded perspective view of an exemplary electronic device according to one or more embodiments;

FIG. 4 is a plan view indicating an exemplary rear cover according to one or more embodiments;

FIG. 5 is a diagram indicating an exemplary electronic device according to one or more embodiments;

FIG. 6 is a diagram indicating an exemplary electronic device according to one or more embodiments;

FIG. 7 is a diagram indicating an exemplary electronic device according to one or more embodiments;

FIG. 8 is a diagram indicating a configuration of an exemplary electronic device according to one or more embodiments;

FIG. 9 is a diagram indicating a configuration of an exemplary electronic device according to one or more embodiments;

FIG. 10 is a diagram indicating an exemplary switch circuit according to one or more embodiments;

FIG. 11 is a graph indicating radiation efficiency of an antenna according to one or more embodiments;

FIG. 12 is a diagram indicating an exemplary electronic device according to one or more embodiments;

FIG. 13 is a diagram indicating an exemplary switch circuit according to one or more embodiments; and

FIG. 14 is a block diagram of an electronic device in a network environment according to various embodiments.

### DETAILED DESCRIPTION

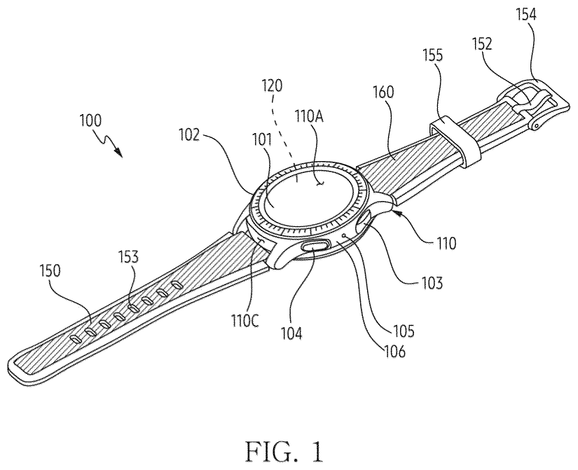

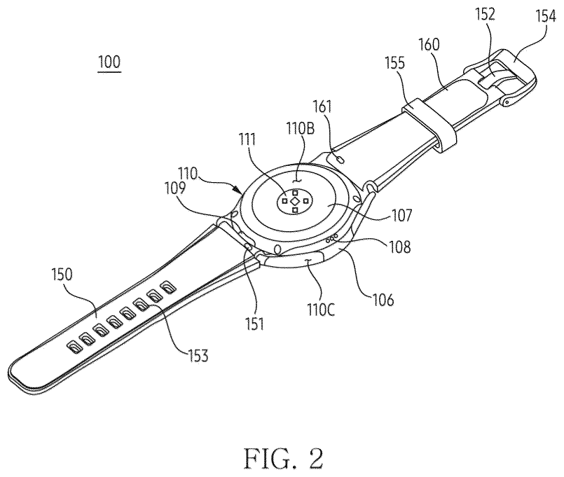

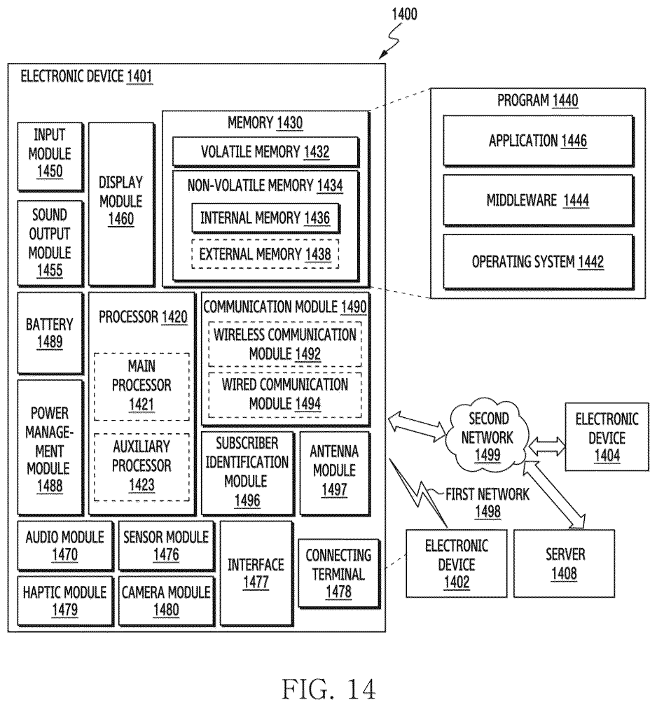

FIG. 1 is a front perspective view of an exemplary electronic device 100 according to one or more embodiments. FIG. 2 is a rear perspective view of an exemplary electronic device 100 according to one or more embodiments. Referring to FIGS. 1 and 2, the electronic device 100 (e.g., an electronic device 1401 of FIG. 14) according to one or more embodiments may include a housing 110 forming a front surface 110A, a rear surface 110B, and a side surface 110C surrounding a space between the front surface 110A and the rear surface 110B, and fastening members 150 and 160 connected to at least a portion of the housing 110 and configured to detachably fasten the electronic device 100 to a part (e.g., a wrist) of a body of a user. For example, the electronic device 100 may be referred to as a wearable device, a wearable electronic device, or an electronic watch.

The housing 110 may refer to a structure forming at least a portion of the front surface 110A, the rear surface 110B, and the side surface 110C. In one or more embodiments, the front surface 110A may be formed by a window 101 (e.g., a glass plate or a polymer plate including various coating layers) of which at least a portion is formed to be substantially transparent. The rear surface 110B may be formed by a substantially opaque rear cover 107. The rear cover 107 may be formed, for example, by coated or colored glass, ceramic, polymer, metal (e.g., aluminum, stainless steel (STS), or magnesium), or a combination of at least two of the above materials. The side surface 110C may be formed by a frame 106 coupled to the window 101 and the rear cover 107. For example, the frame 106 may include a conductive portion (e.g., metal). The conductive portion may be used as an antenna of the electronic device 100. For example, a wireless communication circuit (e.g., a wireless communication module 1492 of FIG. 14) of the electronic device 100 may transmit or receive a radio frequency (RF) signal using the conductive portion of the frame 106. In one or more embodiments, a printed circuit board 380 may at least partially provide a ground of the antenna. For example, a region formed of a conductive material of the printed circuit board 380 may be used as the ground of the antenna. In one or more embodiments, the frame 106 may be referred to as a ‘metal frame’, a ‘side member’, or a ‘side bezel structure’.

The fastening members 150 and 160 may be formed of various materials and shapes. For example, by a woven fabric, leather, rubber, urethane, metal, ceramic, or a combination of at least two of the above materials, the fastening members 150 and 160 may be formed integrally or may be formed such that a plurality of unit links are movable to each other.

According to one or more embodiments, the electronic device 100 may include at least one or more of a display 120, an audio module (e.g., a sound output module 1455 and/or an audio module 1470 of FIG. 14), a sensor module 111, key input devices 102, 103, and 104, and a connector hole 109. In some embodiments, the electronic device 100 may omit at least one of the components (e.g., the key input devices 102, 103, and 104, the connector hole 109, or the sensor module 111) or may additionally include another component.

The display 120 may be exposed, for example, through a significant portion of the window 101. The shape of the display 120 may be a shape corresponding to a shape of the window 101, and may be various shapes such as a circle, an oval, or a polygon. The display 120 may be coupled to or disposed adjacent to a touch detection circuit, a pressure sensor capable of measuring an intensity (pressure) of a touch, and/or a fingerprint sensor.

The audio module may include a microphone hole 105 and a speaker hole 108. A microphone for obtaining external sound may be disposed inside the microphone hole 105. The microphone may include a plurality of microphones to detect a direction of sound, but is not limited thereto. The speaker hole 108 may be used as an external speaker and a call receiver. In some embodiments, the speaker hole 108 and the microphone hole 105 may be implemented as one hole as the speaker hole 108 is integrated to the microphone hole 105, or a speaker without the speaker hole 108 (e.g., a piezo speaker) may be included.

The sensor module 111 may generate an electrical signal or a data value corresponding to an operating state inside the electronic device 100 or an external environmental state. The sensor module 111 may include, for example, the sensor module 111 (e.g., a heart rate monitor (HRM) sensor) disposed on the rear surface 110B of the housing 110. The electronic device 100 may further include at least one of sensor module, for example, a gesture sensor, a gyro sensor, a barometric sensor, a magnetic sensor, an acceleration sensor, a grip sensor, a color sensor, an infrared (IR) sensor, a biometric sensor, a temperature sensor, a humidity sensor, or an illuminance sensor.

In one or more embodiments, the key input devices 102, 103, and 104 may include the wheel key 102 disposed on the front surface 110A of the housing 110 and rotatable in at least one direction and/or the key buttons 103 and 104 disposed on the side surface 110C of the housing 110. For example, the wheel key 102 may have a shape corresponding to the shape of the window 101. In one or more embodiments, the electronic device 100 may not include a portion or all of the key input devices 102, 103, and 104 mentioned above. For example, the electronic device 100 may not include the wheel key 102. In one or more embodiments, the wheel key 102 that is not included may be implemented in another form such as a soft key and the like on the display 120.

The connector hole 109 may accommodate a connector (e.g., a USB connector) for transmitting and receiving power and/or data with an external electronic device; The electronic device 100 may further include, for example, a connector cover covering at least a portion of the connector hole 109 and blocking an inflow of external foreign substances into the connector hole. In another embodiment, the electronic device 100 may not include the connector hole 109, and in this case, the electronic device 100 may transmit and receive power and/or data with the external electronic device using wireless communication.

In one or more embodiments, the fastening members 150 and 160 may be detachably fastened to at least a partial region of the housing 110 using locking members 151 and 161. For example, the fastening members 150 and 160 may include at least one of a fixing member 152, a fixing member fastening hole 153, a band guide member 154, and a band fixing ring 155.

The fixing member 152 may be configured to fix the housing 110 and the fastening members 150 and 160 to a part (e.g., the wrist, and the like) of the body of the user. The fixing member fastening hole 153 may fix the housing 110 and the fastening members 150 and 160 to the part of the body of the user by corresponding to the fixing member 152. The band guide member 154 is configured to limit a movement range of the fixing member 152 when the fixing member 152 is fastened to the fixing member fastening hole 153, such that the fastening members 150 and 160 are fastened in close contact with the part of the body of the user. The band fixing ring 155 may limit a movement range of the fastening members 150 and 160 in a state in which the fixing member 152 and the fixing member fastening hole 153 are fastened.

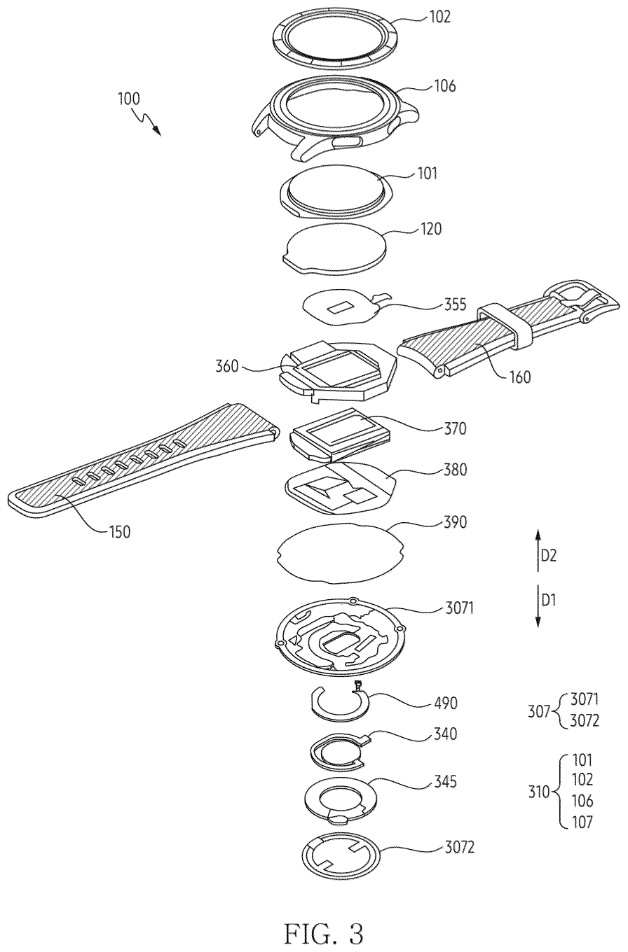

FIG. 3 is an exploded perspective view of an exemplary electronic device 100 according to one or more embodiments. An illustrated first direction D1 may be a direction that is substantially perpendicular to a window 101 and faces a rear cover 307 from the window 101, and a second direction D2 may be a direction opposite to the first direction D1. Hereinafter, overlapping descriptions of configurations having the same reference numerals as the above-described configurations may be omitted.

Referring to FIG. 3, the electronic device 100 may include a housing 310, an antenna 355, a bracket 360, a battery 370, a printed circuit board 380, a sealing member 390, a wireless charging coil 345, and a sensor module 340. The electronic device 100 may further include a conductive member 490 positioned between the printed circuit board 380 and a second member 3072.

In one or more embodiments, the housing 310 (e.g., the housing 110 of FIGS. 1 and 2) may include a wheel key 102, the window 101, the rear cover 307, and a frame 106 forming an exterior (e.g., the front surface 110A, the rear surface 110B, and the side surface 110C of FIGS. 1 and 2) of the electronic device 100. In another embodiment, the housing 310 of the electronic device 100 may not include the wheel key 102, and in this case, the front surface of the electronic device 100 may be formed by the window 101, or the window 101 and the frame 106 may form the front surface together.

In one or more embodiments, the rear cover 307 (e.g., the rear cover 107 of FIGS. 1 and 2) may be disposed on the frame 106. For example, the rear cover 307 may be disposed in the first direction D1 of the frame 106. For example, the rear cover 307 may be coupled to the frame 106. In one or more embodiments, the rear cover 307 may include a first member 3071 and the second member 3072. The second member 3072 may be disposed under the first member 3071 (e.g., in the first direction D1). The first member 3071 may be connected to the frame 106. The second member 3072 may be connected to the first member 3071 to close a hollow formed in the first member 3071.

In one or more embodiments, the bracket 360 may be disposed inside the housing 310. For example, the bracket 360 may be disposed inside the frame 106. The bracket 360 may be positioned between a display 120 and the printed circuit board 380. For example, the display 120 may be disposed on a surface (e.g., a surface facing the second direction D2) of the bracket 360, and the printed circuit board 380 may be disposed on another surface (e.g., a surface facing the first direction D1). The bracket 360 may support the display 120 and the printed circuit board 380. The bracket 360 may be formed of a metal material and/or a non-metal material (e.g., polymer).

In one or more embodiments, the printed circuit board 380 may be equipped with a processor (e.g., a processor 1420 of FIG. 14), memory (e.g., memory 1430 of FIG. 14), and/or an interface (e.g., an interface 1477 of FIG. 14). The processor may include, for example, one or more of a central processing unit, an application processor, a graphic processing unit (GPU), an application processor sensor processor, or a communication processor. The memory may include, for example, volatile memory or non-volatile memory. The interface may include, for example, a high definition multimedia interface (HDMI), a universal serial bus (USB) interface, an SD card interface, and/or an audio interface. The interface may, for example, connect electrically or physically the electronic device 100 to an external electronic device and may include a USB connector, an SD card/MMC connector, or an audio connector.

In one or more embodiments, the battery 370 is a device for supplying power to at least one component of the electronic device 100, and may include, for example, a rechargeable secondary battery. The battery 370 may be accommodated in a space formed in the bracket 360 and positioned between the bracket 360 and the printed circuit board 380.

In one or more embodiments, the antenna 355 may be disposed between the display 120 and the bracket 360. For example, the antenna 355 may be attached to a back surface of the display 120, but is not limited thereto. The antenna 355 may include, for example, a near field communication (NFC) antenna and/or a magnetic secure transmission (MST) antenna. The antenna 355 may, for example, perform short-range communication with an external device or transmit a magnetic-based signal including a short-range communication signal or payment data. In another embodiment, the antenna 355 may be disposed on the printed circuit board 380. For example, the antenna 355 may be formed in a chip shape and mounted on the printed circuit board 380.

In one or more embodiments, the sealing member 390 may be interposed between the frame 106 and the first member 3071 of the rear cover 307. The sealing member 390 may be configured to block moisture and foreign substances flowing between the frame 106 and the first member 3071 from the outside.

In one or more embodiments, the wireless charging coil 345 configured to transmit and receive a power signal with an external device may be disposed between the first member 3071 and the second member 3072. For example, a hollow aligned with a hollow formed in the first member 3071 may be formed in the wireless charging coil 345.

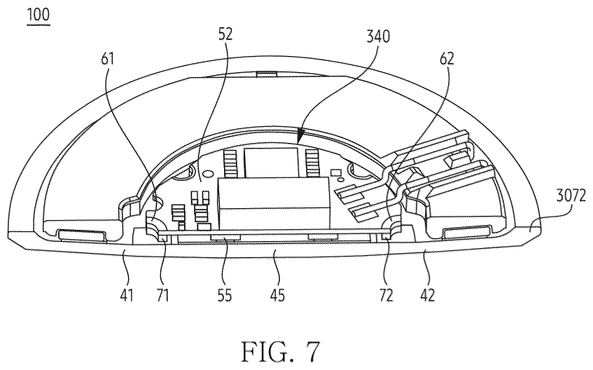

In one or more embodiments, the sensor module 340 may be disposed between the first member 3071 and the second member 3072. The sensor module 340 may be disposed in a form at least partially accommodated in the hollow of the wireless charging coil 345. The sensor module 340 may at least partially face the second member 3072. For example, the sensor module 340 may be disposed to face the second member 3072. The sensor module 340 may detect biometric information of a user through the second member 3072. For example, the sensor module 340 may include an optical sensor (e.g., a sensor 55 of FIG. 7) for detecting a heart rate and/or an oxygen saturation level of the user in contact with the second member 3072. In this case, the second member 3072 may be formed of a material capable of at least partially transmitting light.

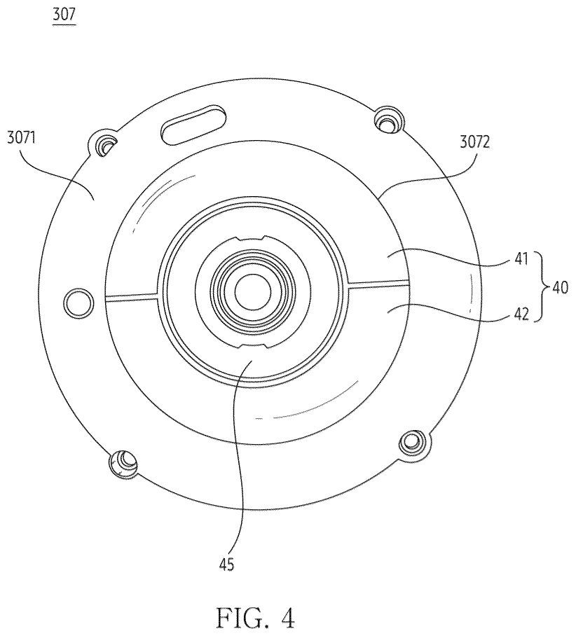

FIG. 4 is a plan view indicating an exemplary rear cover according to one or more embodiments. Referring to FIG. 4, a second member 3072 of a rear cover 307 according to one or more embodiments may include a central region 45. The central region 45 may be aligned with a light emitting unit and a light receiving unit of a sensor module (e.g., the sensor module 340 of FIG. 3). The central region 45 may be configured to be capable of transmitting light by the light emitting unit and the light receiving unit.

In one or more embodiments, the second member 3072 of the rear cover 307 may include an electrode 40. The electrode 40 may include a first electrode 41 and a second electrode 42. For example, the first electrode 41 and the second electrode 42 may at least partially surround the central region 45. The first electrode 41 and the second electrode 42 may include a conductive material. In one or more embodiments, the first electrode 41 and the second electrode 42 may be configured to detect an electrical signal. For example, the first electrode 41 and the second electrode 42 may detect a voltage signal from a body of a user in contact therewith.

In one or more embodiments, a first member 3071 may surround the second member 3072. For example, the second member 3072 may be disposed in the first member 3071. For example, the first member 3071 may surround the first electrode 41 and the second electrode 42 of the second member 3072.

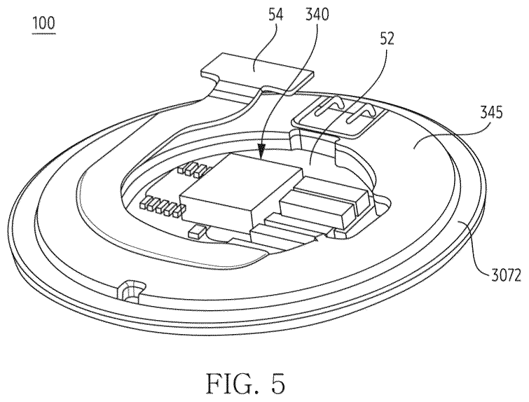

FIG. 5 is a diagram indicating an exemplary electronic device according to one or more embodiments. Referring to FIG. 5, in one or more embodiments, a sensor module 340 and a wireless charging coil 345 may be disposed on a second member 3072. In one or more embodiments, the sensor module 340 may include a substrate 52 and/or a connection member 54. The connection member 54 may include, for example, a flexible printed circuit board. The connection member 54 may connect the substrate 52 to another substrate (e.g., the printed circuit board 380 of FIG. 3).

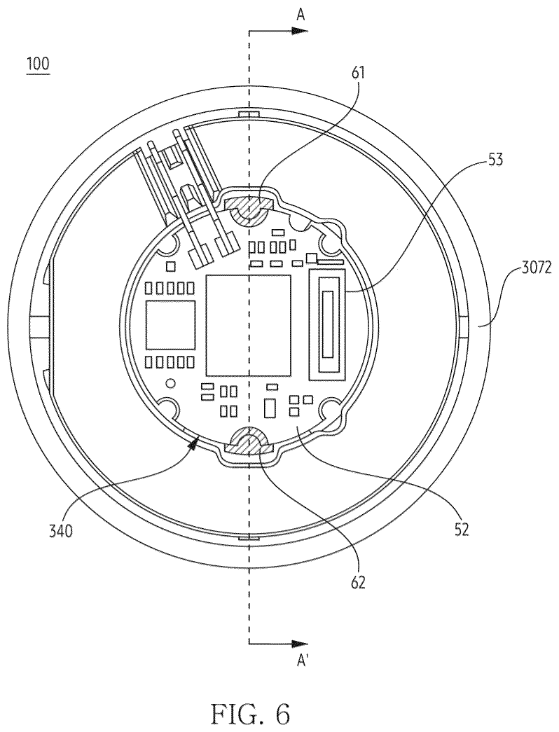

FIG. 6 is a diagram indicating an exemplary electronic device according to one or more embodiments. Referring to FIG. 6, in one or more embodiments, a sensor module 340 may include a connector 53, a first connection portion 61, and a second connection portion 62.

In one or more embodiments, a substrate 52 may include, for example, a printed circuit board. The substrate 52 may be positioned between a printed circuit board (e.g., the printed circuit board 380 of FIG. 3) of an electronic device 100 and a second member 3072.

In one or more embodiments, a connector 53 to which a connection member (e.g., the connection member 54 of FIG. 5) is coupled may be disposed on the substrate 52. In one or more embodiments, the substrate 52 may include the first connection portion 61 and the second connection portion 62.

FIG. 7 is a diagram indicating an exemplary electronic device according to one or more embodiments. FIG. 7 is a perspective view indicating a cross section cut along line A-A′ of FIG. 6. Referring to FIG. 7, in one or more embodiments, a sensor module 340 may include a sensor 55 disposed on a substrate 52 to face a second member 3072. For example, the sensor 55 may include an optical sensor including at least one light emitting unit and at least one light receiving unit, but is not limited thereto. In one or more embodiments, the sensor 55 may be aligned with a central region 45 of a second member 3072. For example, the sensor 55 may be positioned in the central region 45 of the second member 3072.

In one or more embodiments, a first connection portion 61 may be connected to a first electrode 41. For example, the first connection portion 61 may be connected to the first electrode 41 through a conductive connection member. For a non-limiting example, the conductive connection member may include a conductive adhesive material (e.g., a conductive bond) electrically connecting the first connection portion 61 and the first electrode 41 by being interposed between the first connection portion 61 and the first electrode 41. For example, the first connection portion 61 may be bonded to the first electrode 41 through the conductive adhesive material. For a non-limiting example, the conductive connection member may include a screw electrically connecting the first connection portion 61 to the first electrode 41 by directly or indirectly contacting the first connection portion 61 and the first electrode 41. In one or more embodiments, a spacer 71 for maintaining a distance between the sensor 55 and the second member 3072 may be disposed between the first connection portion 61 and the second member 3072. Alternatively, or optionally, the spacer 71 may be formed to have conductivity for the electrical connection between the first connection portion 61 and the first electrode 41.

In one or more embodiments, a second connection portion 62 may be connected to a second electrode 42. For example, the second connection portion 62 may be electrically connected to the second electrode 42 through a conductive adhesive material (e.g., a conductive bond) interposed between the second connection portion 62 and the second electrode 42. For example, the second connection portion 62 may be bonded to the second electrode 42 through the conductive adhesive material. In one or more embodiments, a spacer 72 for maintaining a distance between the sensor 55 and the second member 3072 may be disposed between the second connection portion 62 and the second member 3072. Alternatively, or optionally, the spacer 72 may be formed to have conductivity for the electrical connection between the second connection portion 62 and the second electrode 42.

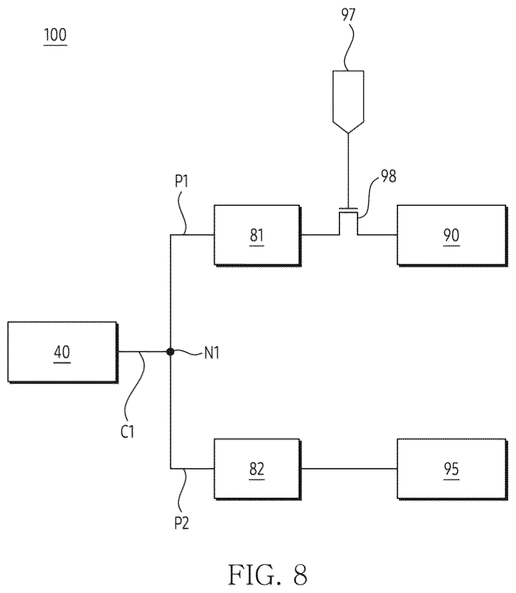

FIG. 8 is a diagram indicating a configuration of an exemplary electronic device according to one or more embodiments. Referring to FIG. 8, an electronic device 100 according to one or more embodiments may include a first electrical path P1, a common path C1, a second electrical path P2, a first circuit 81, a second circuit 82, a sensor circuit 90, and a switch circuit 95.

In one or more embodiments, an electrode 40 may be electrically coupled to the sensor circuit 90 through the first electrical path P1. For example, the first electrical path P1 may include the first circuit 81. The first circuit 81 may be configured to selectively pass an electrical signal (e.g., a bio-signal) detected through the electrode 40. The electrical signal may include, for example, a DC signal or a low frequency signal having a frequency relatively lower than that of an RF signal. For a non-limiting example, the first circuit 81 may include a band pass filter or a low pass filter selectively passing the electrical signal. For a non-limiting example, the first circuit 81 may include a resistor, an inductor, or an RF choke.

The sensor circuit 90 may be connected to a processor (e.g., a processor 1420 of FIG. 14) of the electronic device 100. For example, the sensor circuit 90 may be connected to the processor through another electrical path. For a non-limiting example, the other electrical path may be understood as being included in the first electrical path P1. The sensor circuit 90 may include an analog front-end for processing the electrical signal transmitted through the first circuit 81. The sensor circuit 90 may provide the processed electrical signal to the processor. The processor may obtain biometric information of a user based on the electrical signal. For a non-limiting example, the biometric information may include an electrocardiogram. In one or more embodiments, at least a portion of the sensor circuit 90 may be integrated into the processor, and in this case, the processor may perform substantially the same function as the sensor circuit 90.

In one or more embodiments, the electrode 40 may be electrically coupled to the switch circuit 95 through the second electrical path P2. For example, the second electrical path P2 may include the second circuit 82. The second circuit 82 may be configured to selectively pass the RF signal. For a non-limiting example, the second circuit 82 may include a band pass filter or a high pass filter selectively passing the RF signal. For a non-limiting example, the second circuit 82 may include a capacitor.

In one or more embodiments, the common path C1 may connect the electrode 40 to the first electrical path P1 and the second electrical path P2. For example, a first end of the common path C1 may be connected to the electrode 40, and a second end of the common path C1 may be connected to the first electrical path P1 and the second electrical path P2. The second electrical path P2 may be branched at a point N1 of the first electrical path P1 positioned between the electrode 40 and the first circuit 81. The first circuit 81 and the second circuit 82 may be connected in parallel with respect to the electrode 40. For a non-limiting example, the common path C1 may be understood as being included in the first electrical path P1 and/or the second electrical path P2.

Additionally, or optionally, the electronic device 100 may include a switch 98 for controlling on/off of the first electrical path P1. The switch 98 may be controlled by a micro controller such as a sensor hub.

In one or more embodiments, the switch circuit 95 may be configured to selectively connect the electrode 40 to the ground of the antenna.

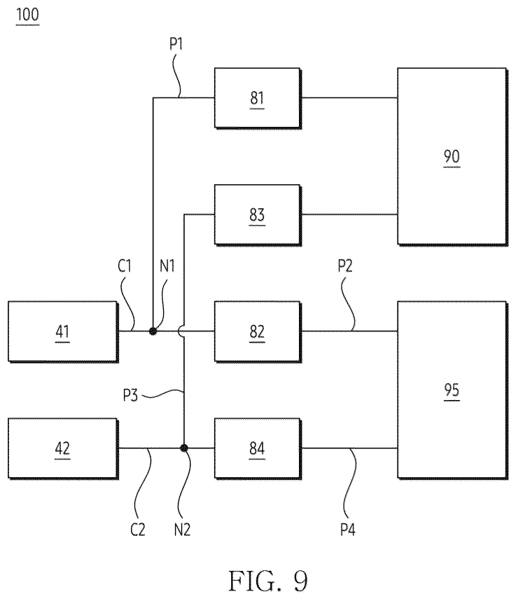

FIG. 9 is a diagram indicating a configuration of an exemplary electronic device according to one or more embodiments. Referring to FIG. 9, an electronic device 100 according to one or more embodiments may include a third electrical path P3, a fourth electrical path P4, a common path C2, a third circuit 83, and a fourth circuit 84.

In one or more embodiments, a first electrode 41 may be electrically coupled to a sensor circuit 90 through a first electrical path P1 including a first circuit 81, and may be electrically coupled to a switch circuit 95 through a second electrical path P2 including a second circuit 82.

In one or more embodiments, a second electrode 42 may be electrically coupled to the sensor circuit 90 through the third electrical path P3. For example, the third electrical path P3 may include the third circuit 83. The third circuit 83 may be configured to selectively pass an electrical signal (e.g., a bio-signal) detected through the second electrode 42. The electrical signal received by the second electrode 42 may include, for example, a low frequency signal having a frequency relatively lower than that of a DC signal or an RF signal. For a non-limiting example, the third circuit 83 may include a band pass filter or a low pass filter selectively passing the electrical signal by the second electrode 42. For a non-limiting example, the third circuit 83 may include a resistor, an inductor, or an RF choke. Additionally, or optionally, the electronic device 100 may include a switch (e.g., the switch 98 of FIG. 9) for controlling on/off of the third electrical path P3.

For example, at least one of the first electrical path P1, the second electrical path P2, the third electrical path P3, and/or the fourth electrical path P4 may include a protection circuit. For example, the protection circuit may include a Zener diode connected to at least one of the first electrical path P1, the second electrical path P2, the third electrical path P3, and/or the fourth electrical path P4, but is not limited thereto.

In one or more embodiments, the processor of the electronic device 100 may determine the biometric information of the user by using electrical signals received through the first electrode 41 and the second electrode 42.

In one or more embodiments, the second electrode 42 may be electrically coupled to the switch circuit 95 through the fourth electrical path P4. The fourth electrical path P4 may include the fourth circuit 84. The fourth circuit 84 may be configured to selectively pass the RF signal. For a non-limiting example, the fourth circuit 84 may include a band pass filter or a high pass filter selectively passing a frequency band of the RF signal. For a non-limiting example, the fourth circuit 84 may include a capacitor.

In one or more embodiments, the common path C2 may connect the second electrode 42 to the third electrical path P3 and the fourth electrical path P4. For example, a first end of the common path C2 may be connected to the second electrode 42, and a second end of the common path C2 may be connected to the third electrical path P3 and the fourth electrical path P4. The fourth electrical path P4 may be branched at a point N2 of the third electrical path P3 positioned between the second electrode 42 and the third circuit 83. The third circuit 83 and the fourth circuit 84 may be connected in parallel with respect to the second electrode 42. For a non-limiting example, the common path C2 may be understood as being included in the third electrical path P3 and/or the fourth electrical path P4.

In one or more embodiments, the second circuit 82 and the third circuit 83 may be at least partially integrated. Accordingly, the second electrical path P2 and the third electrical path P3 may also be at least partially integrated. However, embodiments herein are not limited thereto.

In one or more embodiments, the first circuit 81 and the third circuit 83 may be referred to as a blocking circuit, an AC block circuit, or an RF block circuit. In one or more embodiments, the second circuit 82 and the fourth circuit 84 may be referred to as a blocking circuit or a DC block circuit.

Referring together to FIG. 7, in one or more embodiments, the first circuit 81, the second circuit 82, the third circuit 83, the fourth circuit 84, the sensor circuit 90, and the switch circuit 95 may be provided to the substrate 52 of the sensor module 340. For example, the first circuit 81, the second circuit 82, the third circuit 83, the fourth circuit 84, the sensor circuit 90, and the switch circuit 95 may be disposed on the substrate 52 of the sensor module 340. Alternatively, at least one of the first circuit 81, the second circuit 82, the third circuit 83, the fourth circuit 84, the sensor circuit 90, and/or the switch circuit 95 may be disposed on another substrate (e.g., the printed circuit board 380 of FIG. 3) distinct from the substrate 52. For example, referring together to FIGS. 5, 6, and 12, the first circuit 81, the third circuit 83, and the sensor circuit 90 may be disposed on the substrate 52, and the processor may be disposed on the printed circuit board 380. In this case, the first electrical path P1 (or the third electrical path P3) connected to the first electrode 41 (or the second electrode 42) may include a first path extending from the first connection portion 61 (or the second connection portion 62) to the first circuit 81 (or the third circuit 83) and a second path extending from the first circuit 81 (or the third circuit 83) to the sensor circuit 90. The other electrical path may include a third path extending from the sensor circuit 90 to the connector 53 of the substrate 52, a fourth path extending from the connector 53 of the substrate 52 to a connector 1220 of the printed circuit board 380, and a fifth path extending from the connector 1220 of the printed circuit board 380 to the processor.

For example, the first circuit 81 and the third circuit 83 may be disposed on the substrate 52, and the sensor circuit 90 may be disposed on the printed circuit board 380. In this case, the first electrical path P1 (or the third electrical path P3) connected to the first electrode 41 (or the second electrode 42) may include the first path extending from the first connection portion 61 (or the second connection portion 62) to the first circuit 81 (or the third circuit 83), a third path extending from the first circuit 81 (or the third circuit 83) to the connector 53 of the substrate 52, the fourth path extending from the connector 53 of the substrate 52 to the connector 1220 of the printed circuit board 380, and a fifth path extending from the connector 1220 of the printed circuit board 380 to the sensor circuit 90. For example, the second circuit 82 and the fourth circuit 84 may be disposed on the substrate 52, and the switch circuit 95 may be disposed on the printed circuit board 380. In this case, the second electrical path P2 (or the fourth electrical path P4) may include a first path extending from the first connection portion 61 (or the second connection portion 62) to the second circuit 82 (or the fourth circuit 84), a second path extending from the second circuit 82 (or the fourth circuit 84) to the connector 53 of the substrate 52, a third path extending from the connector 53 of the substrate 52 to the connector 1220 of the printed circuit board 380, and a fourth path extending from the connector 1220 of the printed circuit board 380 to the switch circuit 95. The first path and the second path may be formed of a conductive material provided by the substrate 52. The third path may be formed of a conductive material provided by the connection member 54. The fourth path may be formed of a conductive material provided by the printed circuit board 380.

However, a configuration providing the first electrical path P1, the second electrical path P2, the third electrical path P3, and the fourth electrical path P4 is not limited to the above-described example, and may vary according to positions where the first circuit 81, the second circuit 82, the third circuit 83, the fourth circuit 84, the sensor circuit 90, and the switch circuit 95 are disposed.

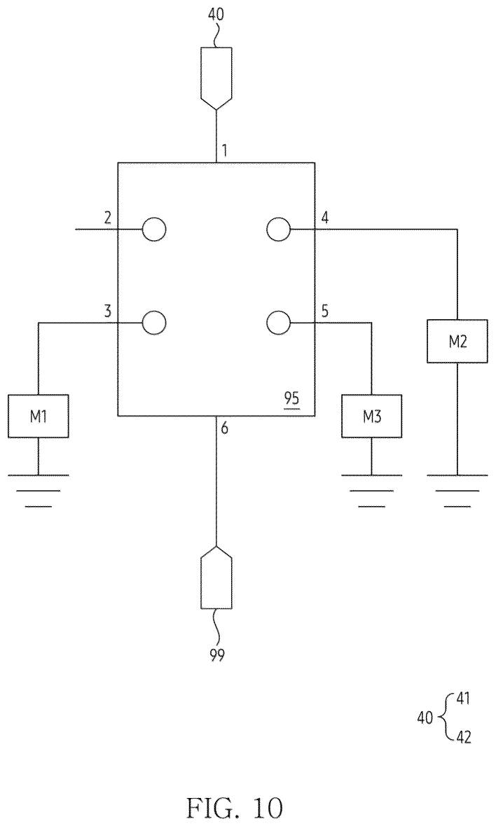

FIG. 10 is a diagram indicating an exemplary switch circuit according to one or more embodiments. Referring to FIG. 10, a switch circuit 95 may include a first pin (or terminal) 1, a second pin 2, a third pin 3, a fourth pin 4, a fifth pin 5, and/or a sixth pin 6.

In one or more embodiments, the switch circuit 95 may be electrically connected to an electrode 40 and a ground of an antenna. For example, the first pin 1 of the switch circuit 95 may be connected to the electrode 40. For example, the first pin 1 may be connected to a first electrode 41 through a second electrical path P2 and/or may be connected to a second electrode 42 through a fourth electrical path P4. For example, the third pin 3 may be connected to the ground of the antenna through a first matching circuit M1. For example, the fourth pin 4 may be connected to the ground of the antenna through a second matching circuit M2. For example, the fifth pin 5 may be connected to the ground of the antenna through a third matching circuit M3. In one or more embodiments, the first matching circuit M1, the second matching circuit M2, and/or the third matching circuit M3 may be configured such that the electrode 40 and the ground of the antenna connected to each other have different electrical lengths. For a non-limiting example, the first matching circuit M1, the second matching circuit M2, and the third matching circuit M3 may include lumped elements (e.g., an inductor and/or a capacitor) having different values. In one or more embodiments, the first matching circuit M1, the second matching circuit M2, and/or the third matching circuit M3 may be omitted.

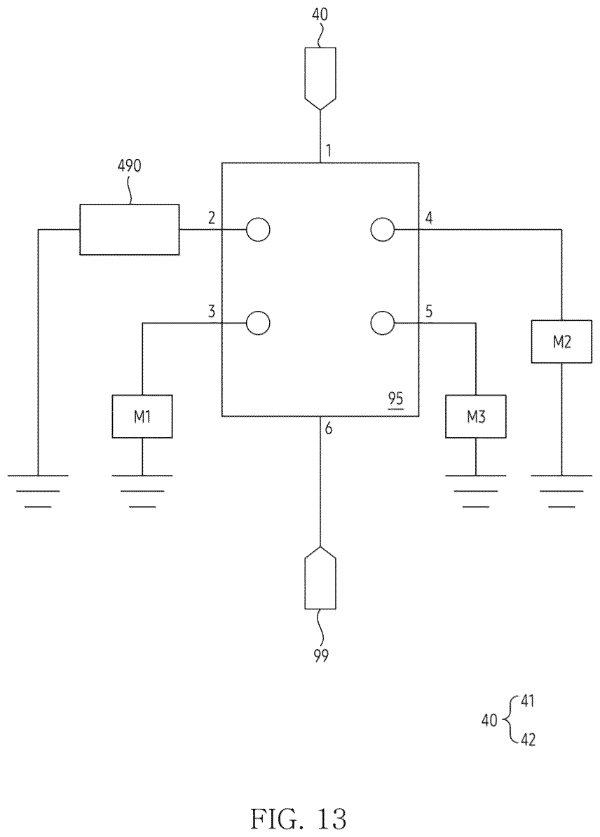

In one or more embodiments, the second pin 2 may be opened, but is not limited thereto. For example, the second pin 2 may be connected to the ground of the antenna. In this case, selectively, the second pin 2 may be connected to the ground of the antenna through a matching circuit. For another example, as illustrated in FIG. 13, the second pin 2 may be connected to a conductive member 490.

In one or more embodiments, a control signal 99 of the switch circuit 95 may be inputted to the sixth pin 6. The control signal 99 may be provided by, for example, a communication processor (e.g., a processor 1420 of FIG. 14) or a wireless communication circuit (e.g., a wireless communication module 1492 of FIG. 14) of an electronic device 100. In one or more embodiments, the first pin 1 of the switch circuit 95 may be connected to at least one of the second to fifth pins 2, 3, 4, and 5.

In one or more embodiments, the switch circuit 95 may be configured to selectively connect the electrode 40 (e.g., the first electrode 41 and/or the second electrode 42) to the ground of the antenna. The communication processor or the wireless communication circuit may control the switch circuit 95 so that the electrode 40 is selectively connected to the ground of the antenna.

In one or more embodiments, the switch circuit 95 may be configured to selectively connect the first electrode 41 and/or the second electrode 42 to the ground of the antenna. For example, the first electrode 41 and the second electrode 42 may be connected to the first pin 1. In this case, the first pin 1 may be connected to at least one of the second to fifth pins 2, 3, 4, and 5. For another example, the first electrode 41 may be connected to the first pin 1, and the second electrode 42 may be connected to the second pin 2. In this case, the switch circuit 95 may connect the first pin 1 and/or the second pin 2 to at least one of the third to fifth pins 3, 4, and 5. However, a configuration of the switch circuit 95 for selectively connecting the first electrode 41 and/or the second electrode 42 to the ground of the antenna is not limited to the above-described example, and various modifications may be possible.

In one or more embodiments, the first electrode 41 and/or the second electrode 42 may be connected to the ground of the antenna. The ground of the antenna may be expanded by the first electrode 41 and the second electrode 42, and accordingly, performance of the antenna may be improved. Together with this, the biometric information may be obtained using the first electrode 41 and the second electrode 42. In addition, the first electrode 41 and/or the second electrode 42 may be connected to the ground of the antenna through at least one of the first matching circuit M1, the second matching circuit M2, or the third matching circuit M3. Through this, wireless communication may be performed adaptively according to required antenna performance.

In another embodiment, the first electrode 41 and/or the second electrode 42 may be used as a radiator of an antenna. In this case, a feeding line connected to the first electrode 41 and/or the second electrode 42 may be formed on a substrate (e.g., the substrate 52 of FIG. 7), and the wireless communication circuit may transmit and receive an RF signal through the feeding line.

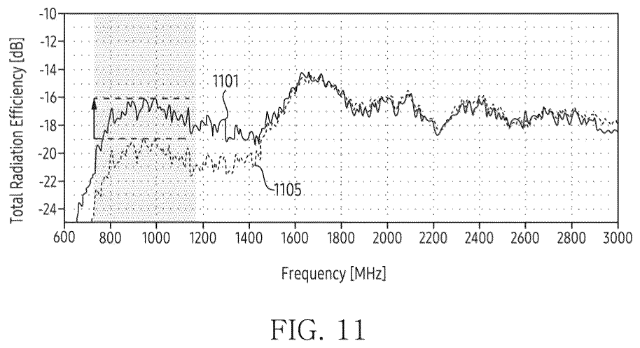

FIG. 11 is a graph indicating radiation efficiency of an antenna according to one or more embodiments. A graph plot 1101 of FIG. 11 indicates radiation efficiency of an electronic device 100 according to one or more embodiments, and a graph plot 1105 indicates radiation efficiency of a device according to a comparative example. In the case of the electronic device 100 according to one or more embodiments, a first electrode 41 and a second electrode 42 may be electrically connected to the ground of the antenna, whereas the device of a comparative example may not be. Referring to FIG. 11, in a frequency band of approximately 700 MHz to 1450 MHz, the radiation efficiency of the graph 1101 may be higher than that of the graph 1105. This may be because a ground area of the antenna of the electronic device 100 according to one or more embodiments is expanded by the first electrode 41 and the second electrode 42. In order to improve a display area, an area of an inactive region (e.g., a black matrix) of a display (e.g., the display 120 of FIG. 3) may be reduced. In this case, performance of the antenna (e.g., a Mid band of approximately 1 GHz to 2.3 GHz) using a slit between the inactive region of the display and a frame (e.g., the frame 106 of FIG. 3) may be degraded. To prevent this, a conductive layer (e.g., a shielding sheet) of the display that interferes with radiation of the slit may be partially removed. However, this may degrade the performance of the antenna (e.g., a Low band of approximately 1 GHz or less) by reducing the ground area of the antenna. The ground area may be secured through a separate conductive member (e.g., a conductive member 490), but a space and a cost may be wasted. In contrast, the electronic device 100 according to one or more embodiments may improve the performance of the antenna by expanding the ground of the antenna by utilizing an electrode 40 for obtaining biometric information.

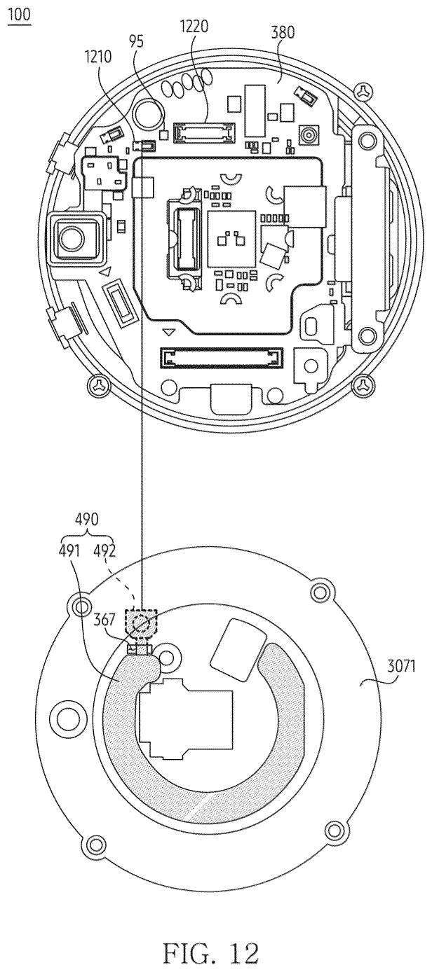

FIG. 12 is a diagram indicating an exemplary electronic device according to one or more embodiments. Referring to FIG. 12, in one or more embodiments, a connector 1220, a switch circuit 95, and a connector 1210 may be disposed on a printed circuit board 380. The connection member 54 (of FIG. 5) may be connected to the connector 1220. The connector 1210 may be electrically connected to the ground of the antenna and the switch circuit 95.

In one or more embodiments, a conductive member 490 may include a first portion 491 and a second portion 492 extending from the first portion 491. The first portion 491 of the conductive member 490 may be disposed on a surface of a first member 3071. The second portion 492 of the conductive member 490 may pass through a hole 367 formed in the first member 3071 and be positioned on another surface (e.g., a surface facing the printed circuit board 380) of the first member 3071. For a non-limiting example, the conductive member 490 may include a conductive sheet, a conductive film, or a conductive pattern provided to a flexible printed circuit board.

In one or more embodiments, the second portion 492 of the conductive member 490 may be electrically connected by contacting the connector 1210. The conductive member 490 may be electrically connected to the ground of the antenna and the switch circuit 95 through the connector 1210.

FIG. 13 is a diagram indicating an exemplary switch circuit according to one or more embodiments. Referring to FIG. 13, a second pin 2 of a switch circuit 95 may be electrically connected to a conductive member 490. The second pin 2 may be electrically connected to the ground of the antenna through the conductive member 490. Through the switch circuit 95, a first electrode 41 and/or a second electrode 42 may be electrically connected to the ground of the antenna through the conductive member 490. Through this, the ground area of the antenna may be further increased.

According to one or more embodiments, a wearable device (e.g., the electronic device 100 of FIG. 1) may comprise a frame (e.g., the frame 106 of FIG. 3) including a conductive portion used as an antenna, a cover (e.g., the rear cover 307 of FIG. 4) coupled to the frame and including an electrode (e.g., the electrode 40 of FIG. 4) configured to detect an electrical signal from a human body when wearing the wearable device, a wireless communication circuit (e.g., the wireless communication module 1492 of FIG. 14) configured to transmit or receive a radio frequency (RF) signal using the antenna, a processor (e.g., the processor 1420 of FIG. 14) configured to obtain biometric information of a user by using the electrical signal, and a switch circuit (e.g., the switch circuit 95 of FIG. 8) connected to a ground of the antenna. The electrode may be coupled to the processor through a first electrical path (e.g., the first electrical path P1 of FIG. 8) comprising a first circuit (e.g., the first circuit 81 of FIG. 8) configured to selectively pass the electrical signal, and coupled to the switch circuit through a second electrical path (e.g., the second electrical path P2 of FIG. 8) comprising a second circuit (e.g., the second circuit 82 of FIG. 8) configured to selectively pass the RF signal. The processor may be configured to control the switch circuit such that the electrode is selectively connected to the ground of the antenna.

In one or more embodiments, the first electrical path and the second electrical path may include a common path (e.g., the common path C1 of FIG. 8) connected to the electrode.

In one or more embodiments, the electrical signal may be a first electrical signal, the electrode may be a first electrode (e.g., the first electrode 41 of FIG. 9), and the cover may include a second electrode (e.g., the second electrode 42 of FIG. 9) configured to detect a second electrical signal. The second electrode may be coupled to the processor through a third electrical path (e.g., the third electrical path P3 of FIG. 9) comprising a third circuit (e.g., the third circuit 83 of FIG. 9) configured to selectively pass the second electrical signal. The second electrode may be coupled to the switch circuit through a fourth electrical path (e.g., the fourth electrical path P4 of FIG. 9) comprising a fourth circuit (e.g., the fourth circuit 84 of FIG. 9) configured to selectively pass the RF signal.

In one or more embodiments, the processor may be configured to obtain biometric information of the user using the first electrical signal and the second electrical signal. The processor may be configured to control the switch circuit such that at least one of the first electrode and the second electrode is selectively connected to the ground of the antenna.

In one or more embodiments, the first electrical path and the second electrical path may include a common path (e.g., the common path C1 of FIG. 9) connected to the first electrode. The third electrical path and the fourth electrical path may include a common path (e.g., the common path C2 of FIG. 9) connected to the second electrode.

According to one or more embodiments; the wearable device may comprise a first printed circuit board (e.g., the printed circuit board 380 of FIG. 3) on which the processor and the switch circuit are disposed, a second printed circuit board (e.g., the substrate 52 of FIG. 5), positioned between the first printed circuit board and the cover, a flexible printed circuit board (e.g., the connection member 54 of FIG. 5) connecting the second printed circuit board to the first printed circuit board, and an optical sensor (e.g., the sensor 55 of FIG. 7) disposed on the second printed circuit board to face the cover.

In one or more embodiments, the second printed circuit board may include a first connection portion (e.g., the first connection portion 61 of FIG. 7) electrically connected to the first electrode and a second connection portion (e.g., the second connection portion 62 of FIG. 7) electrically connected to the second electrode.

According to one or more embodiments, the wearable device may comprise a first conductive adhesive member electrically connecting the first connection portion and the first electrode, and a second conductive adhesive member electrically connecting the second connection portion and the first electrode.

In one or more embodiments, the first circuit may be provided to the first printed circuit board or the second printed circuit board. The second circuit may be provided to the first printed circuit board or the second printed circuit board. The third circuit may be provided to the first printed circuit board or the second printed circuit board. The fourth circuit may be provided to the first printed circuit board or the second printed circuit board.

According to one or more embodiments; the wearable device may comprise a sensor circuit (e.g., the sensor circuit 90 of FIG. 8) configured to process the first electrical signal and the second electrical signal. The sensor circuit may be disposed on the second printed circuit board.

In one or more embodiments, the sensor circuit may be provided to the first printed circuit board or the second printed circuit board.

In one or more embodiments; the processor may include at least one sensor circuit (e.g., the sensor circuit 90 of FIG. 8) configured to process the first electrical signal and the second electrical signal.

In one or more embodiments, a conductive member (e.g., the conductive member 490 of FIG. 11) positioned between the first printed circuit board and the second printed circuit board may be included. The conductive member may be connected to the switch circuit and the ground of the antenna.

In one or more embodiments, the switch circuit may comprise a first pin (e.g., the first pin 1 of FIG. 13) to which the first electrode is connected, a second pin (e.g., the second pin 2 of FIG. 13) to which the conductive member is connected, and a third pin (e.g., the third pin 3 of FIG. 13) connected to the ground of the antenna. The switch circuit may be configured such that the first pin is selectively connected to at least one of the second pin and the third pin.

In one or more embodiments, the switch circuit may include a fourth pin (e.g., the fourth pin 4 of FIG. 13) connected to the ground of the antenna through at least one lumped element. The switch circuit may be configured such that the first pin is selectively connected to at least one of the second pin, the third pin, and the fourth pin.

In one or more embodiments, the third pin may be connected to the ground of the antenna through at least one other lumped element.

In one or more embodiments, the first circuit may include a resistor or an inductor. The second circuit may include a capacitor.

In one or more embodiments, the biometric information includes an electrocardiogram.

According to one or more embodiments, an electronic watch (e.g., the electronic device 100 of FIG. 1) may comprise a housing including a first cover (e.g., the window 101 of FIG. 3), a second cover (e.g., the rear cover 307 of FIG. 3), and a metal frame (e.g., the frame 106 of FIG. 3) disposed between the first cover and the second cover and used as an antenna for a radio frequency (RF) signal. The second cover may include a first electrode (e.g., the first electrode 41 of FIG. 4) and a second electrode (e.g., the second electrode 42 of FIG. 4) that are configured to detect an electrical signal. The electronic watch may comprise at least one first circuit configured to selectively pass the electrical signal, at least one second circuit configured to selectively pass the RF signal, a sensor circuit (e.g., the sensor circuit 90 of FIG. 9) coupled to the first electrode and the second electrode through the at least one first circuit and configured to process the detected electrical signal, a switch circuit (e.g., the switch circuit 95 of FIG. 9) coupled to the first electrode and the second electrode through the at least one second circuit, and connected to a ground of the antenna, and at least one processor (e.g., the processor 1420 of FIG. 14) operatively connected to the metal frame, the sensor circuit, and the switch circuit. The at least one processor may be configured to transmit or receive the RF signal using the antenna. The at least one processor may be configured to obtain biometric information of a user using the electrical signal provided from the sensor circuit. The at least one processor may be configured to control the switch circuit such that at least one of the first electrode and the second electrode is selectively connected to the ground of the antenna.

According to one or more embodiments, the electronic watch may comprise a conductive member (e.g., the conductive member 490 of FIG. 12) disposed within the housing and electrically connected to the switch circuit. The at least one processor may be configured to control the switch circuit such that at least one of the first electrode and the second electrode is selectively connected to the conductive member or the ground of the antenna.

In one or more embodiments, the at least one first circuit may include a first element and a second element. The at least one second circuit may include a third element and a fourth element. The first element and the third element may be connected in parallel to the first electrode. The second element and the fourth element may be connected in parallel to the second electrode.

FIG. 14 is a block diagram illustrating an electronic device 1401 in a network environment 1400 according to various embodiments. Referring to FIG. 14, the electronic device 1401 in the network environment 1400 may communicate with an electronic device 1402 via a first network 1498 (e.g., a short-range wireless communication network), or at least one of an electronic device 1404 or a server 1408 via a second network 1499 (e.g., a long-range wireless communication network). According to one or more embodiments, the electronic device 1401 may communicate with the electronic device 1404 via the server 1408. According to one or more embodiments, the electronic device 1401 may include a processor 1420, memory 1430, an input module 1450, a sound output module 1455, a display module 1460, an audio module 1470, a sensor module 1476, an interface 1477, a connecting terminal 1478, a haptic module 1479, a camera module 1480, a power management module 1488, a battery 1489, a communication module 1490, a subscriber identification module (SIM) 1496, or an antenna module 1497. In some embodiments, at least one of the components (e.g., the connecting terminal 1478) may be omitted from the electronic device 1401, or one or more other components may be added in the electronic device 1401. In some embodiments, some of the components (e.g., the sensor module 1476, the camera module 1480, or the antenna module 1497) may be implemented as a single component (e.g., the display module 1460).

The processor 1420 may execute, for example, software (e.g., a program 1440) to control at least one other component (e.g., a hardware or software component) of the electronic device 1401 coupled with the processor 1420, and may perform various data processing or computation. According to one or more embodiments, as at least part of the data processing or computation, the processor 1420 may store a command or data received from another component (e.g., the sensor module 1476 or the communication module 1490) in volatile memory 1432, process the command or the data stored in the volatile memory 1432, and store resulting data in non-volatile memory 1434. According to one or more embodiments, the processor 1420 may include a main processor 1421 (e.g., a central processing unit (CPU) or an application processor (AP)), or an auxiliary processor 1423 (e.g., a graphics processing unit (GPU), a neural processing unit (NPU), an image signal processor (ISP), a sensor hub processor, or a communication processor (CP)) that is operable independently from, or in conjunction with, the main processor 1421. For example, when the electronic device 1401 includes the main processor 1421 and the auxiliary processor 1423, the auxiliary processor 1423 may be adapted to consume less power than the main processor 1421, or to be specific to a specified function. The auxiliary processor 1423 may be implemented as separate from, or as part of the main processor 1421.

The auxiliary processor 1423 may control at least some of functions or states related to at least one component (e.g., the display module 1460, the sensor module 1476, or the communication module 1490) among the components of the electronic device 1401, instead of the main processor 1421 while the main processor 1421 is in an inactive (e.g., sleep) state, or together with the main processor 1421 while the main processor 1421 is in an active state (e.g., executing an application). According to one or more embodiments, the auxiliary processor 1423 (e.g., an image signal processor or a communication processor) may be implemented as part of another component (e.g., the camera module 1480 or the communication module 1490) functionally related to the auxiliary processor 1423. According to one or more embodiments, the auxiliary processor 1423 (e.g., the neural processing unit) may include a hardware structure specified for artificial intelligence model processing. An artificial intelligence model may be generated by machine learning. Such learning may be performed, e.g., by the electronic device 1401 where the artificial intelligence is performed or via a separate server (e.g., the server 1408). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 1430 may store various data used by at least one component (e.g., the processor 1420 or the sensor module 1476) of the electronic device 1401. The various data may include, for example, software (e.g., the program 1440) and input data or output data for a command related thereto. The memory 1430 may include the volatile memory 1432 or the non-volatile memory 1434.

The program 1440 may be stored in the memory 1430 as software, and may include, for example, an operating system (OS) 1442, middleware 1444, or an application 1446.

The input module 1450 may receive a command or data to be used by another component (e.g., the processor 1420) of the electronic device 1401, from the outside (e.g., a user) of the electronic device 1401. The input module 1450 may include, for example, a microphone, a mouse, a keyboard, a key (e.g., a button), or a digital pen (e.g., a stylus pen).

The sound output module 1455 may output sound signals to the outside of the electronic device 1401. The sound output module 1455 may include, for example, a speaker or a receiver. The speaker may be used for general purposes, such as playing multimedia or playing record. The receiver may be used for receiving incoming calls. According to one or more embodiments, the receiver may be implemented as separate from, or as part of the speaker.

The display module 1460 may visually provide information to the outside (e.g., a user) of the electronic device 1401. The display module 1460 may include, for example, a display, a hologram device, or a projector and control circuitry to control a corresponding one of the display, hologram device, and projector. According to one or more embodiments, the display module 1460 may include a touch sensor adapted to detect a touch, or a pressure sensor adapted to measure the intensity of force incurred by the touch.

The audio module 1470 may convert a sound into an electrical signal and vice versa. According to one or more embodiments, the audio module 1470 may obtain the sound via the input module 1450, or output the sound via the sound output module 1455 or a headphone of an external electronic device (e.g., an electronic device 1402) directly (e.g., wiredly) or wirelessly coupled with the electronic device 1401.

The sensor module 1476 may detect an operational state (e.g., power or temperature) of the electronic device 1401 or an environmental state (e.g., a state of a user) external to the electronic device 1401, and then generate an electrical signal or data value corresponding to the detected state. According to one or more embodiments, the sensor module 1476 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 1477 may support one or more specified protocols to be used for the electronic device 1401 to be coupled with the external electronic device (e.g., the electronic device 1402) directly (e.g., wiredly) or wirelessly. According to one or more embodiments, the interface 1477 may include, for example, a high definition multimedia interface (HDMI), a universal serial bus (USB) interface, a secure digital (SD) card interface, or an audio interface.

A connecting terminal 1478 may include a connector via which the electronic device 1401 may be physically connected with the external electronic device (e.g., the electronic device 1402). According to one or more embodiments, the connecting terminal 1478 may include, for example, an HDMI connector, a USB connector, an SD card connector, or an audio connector (e.g., a headphone connector).

The haptic module 1479 may convert an electrical signal into a mechanical stimulus (e.g., a vibration or a movement) or electrical stimulus which may be recognized by a user via his tactile sensation or kinesthetic sensation. According to one or more embodiments, the haptic module 1479 may include, for example, a motor, a piezoelectric element, or an electric stimulator.

The camera module 1480 may capture a still image or moving images. According to one or more embodiments, the camera module 1480 may include one or more lenses, image sensors, image signal processors, or flashes.

The power management module 1488 may manage power supplied to the electronic device 1401. According to one or more embodiments, the power management module 1488 may be implemented as at least part of, for example, a power management integrated circuit (PMIC).

The battery 1489 may supply power to at least one component of the electronic device 1401. According to one or more embodiments, the battery 1489 may include, for example, a primary cell which is not rechargeable, a secondary cell which is rechargeable, or a fuel cell.

The communication module 1490 may support establishing a direct (e.g., wired) communication channel or a wireless communication channel between the electronic device 1401 and the external electronic device (e.g., the electronic device 1402, the electronic device 1404, or the server 1408) and performing communication via the established communication channel. The communication module 1490 may include one or more communication processors that are operable independently from the processor 1420 (e.g., the application processor (AP)) and supports a direct (e.g., wired) communication or a wireless communication. According to one or more embodiments, the communication module 1490 may include a wireless communication module 1492 (e.g., a cellular communication module, a short-range wireless communication module, or a global navigation satellite system (GNSS) communication module) or a wired communication module 1494 (e.g., a local area network (LAN) communication module or a power line communication (PLC) module). A corresponding one of these communication modules may communicate with the external electronic device via the first network 1498 (e.g., a short-range communication network, such as Bluetooth™, wireless-fidelity (Wi-Fi) direct, or infrared data association (IrDA)) or the second network 1499 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 1492 may identify and authenticate the electronic device 1401 in a communication network, such as the first network 1498 or the second network 1499, using subscriber information (e.g., international mobile subscriber identity (IMSI)) stored in the subscriber identification module 1496.

The wireless communication module 1492 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 1492 may support a high-frequency band (e.g., the mmWave band) to achieve, e.g., a high data transmission rate. The wireless communication module 1492 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 1492 may support various requirements specified in the electronic device 1401, an external electronic device (e.g., the electronic device 1404), or a network system (e.g., the second network 1499). According to one or more embodiments, the wireless communication module 1492 may support a peak data rate (e.g., 20 Gbps or more) for implementing eMBB, loss coverage (e.g., 1464 dB or less) for implementing mMTC, or U-plane latency (e.g., 0.5 ms or less for each of downlink (DL) and uplink (UL), or a round trip of 14 ms or less) for implementing URLLC.

The antenna module 1497 may transmit or receive a signal or power to or from the outside (e.g., the external electronic device) of the electronic device 1401. According to one or more embodiments, the antenna module 1497 may include an antenna including a radiating element composed of a conductive material or a conductive pattern formed in or on a substrate (e.g., a printed circuit board (PCB)). According to one or more embodiments, the antenna module 1497 may include a plurality of antennas (e.g., array antennas). In such a case, at least one antenna appropriate for a communication scheme used in the communication network, such as the first network 1498 or the second network 1499, may be selected, for example, by the communication module 1490 (e.g., the wireless communication module 1492) from the plurality of antennas. The signal or the power may then be transmitted or received between the communication module 1490 and the external electronic device via the selected at least one antenna. According to one or more embodiments, another component (e.g., a radio frequency integrated circuit (RFIC)) other than the radiating element may be additionally formed as part of the antenna module 1497.

According to various embodiments, the antenna module 1497 may form a mmWave antenna module. According to one or more embodiments, the mmWave antenna module may include a printed circuit board, an RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to one or more embodiments, commands or data may be transmitted or received between the electronic device 1401 and the external electronic device 1404 via the server 1408 coupled with the second network 1499. Each of the electronic devices 1402 or 1404 may be a device of a same type as, or a different type, from the electronic device 1401. According to one or more embodiments, all or some of operations to be executed at the electronic device 1401 may be executed at one or more of the external electronic devices 1402, 1404, or 1408. For example, if the electronic device 1401 should perform a function or a service automatically, or in response to a request from a user or another device, the electronic device 1401, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 1401. The electronic device 1401 may provide the outcome, with or without further processing of the outcome, as at least part of a reply to the request. To that end, a cloud computing, distributed computing, mobile edge computing (MEC), or client-server computing technology may be used, for example. The electronic device 1401 may provide ultra low-latency services using, e.g., distributed computing or mobile edge computing. In another embodiment, the external electronic device 1404 may include an internet-of-things (IoT) device. The server 1408 may be an intelligent server using machine learning and/or a neural network. According to one or more embodiments, the external electronic device 1404 or the server 1408 may be included in the second network 1499. The electronic device 1401 may be applied to intelligent services (e.g., smart home, smart city, smart car, or healthcare) based on 5G communication technology or IoT-related technology.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to one or more embodiments of the disclosure, the electronic devices are not limited to those described above.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” or “connected with” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to one or more embodiments, the module may be implemented in a form of an application-specific integrated circuit (ASIC).

Various embodiments as set forth herein may be implemented as software (e.g., the program 1440) including one or more instructions that are stored in a storage medium (e.g., internal memory 1436 or external memory 1438) that is readable by a machine (e.g., the electronic device 1401). For example, a processor (e.g., the processor 1420) of the machine (e.g., the electronic device 1401) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between a case in which data is semi-permanently stored in the storage medium and a case in which the data is temporarily stored in the storage medium.

According to one or more embodiments, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. A wearable device comprising:
a frame comprising a conductive portion that is an antenna;
a cover coupled to the frame and comprising an electrode configured to detect an electrical signal from a body of a user when the user is wearing the wearable device;
a wireless communication circuit configured to any of transmit and receive a radio frequency (RF) signal from the antenna;
a processor configured to obtain biometric information of the user by using the electrical signal; and
a switch circuit connected to a ground of the antenna, and
wherein the electrode is:
coupled to the processor through a first electrical path comprising a first circuit configured to selectively pass the electrical signal; and
coupled to the switch circuit through a second electrical path comprising a second circuit configured to selectively pass the RF signal, and
wherein the processor is further configured to control the switch circuit to selectively connect the electrode to the ground of the antenna.

2. The wearable device of claim 1,
wherein the first electrical path and the second electrical path comprise a common path connected to the electrode.

3. The wearable device of claim 1, wherein
the electrical signal is a first electrical signal,
the electrode is a first electrode,
the cover comprises a second electrode configured to detect a second electrical signal, and
the second electrode is:
coupled to the processor through a third electrical path comprising a third circuit configured to selectively pass the second electrical signal; and
coupled to the switch circuit through a fourth electrical path comprising a fourth circuit configured to selectively pass the RF signal.

4. The wearable device of claim 3, wherein
the processor is further configured to:
obtain the biometric information of the user based on the first electrical signal and the second electrical signal; and
control the switch circuit to selectively connect at least one of the first electrode and the second electrode to the ground of the antenna.

5. The wearable device of claim 4, wherein
the first electrical path and the second electrical path comprise a first common path connected to the first electrode, and
the third electrical path and the fourth electrical path comprise a second common path connected to the second electrode.

6. The wearable device of claim 3, further comprising:
a first printed circuit board on which the processor and the switch circuit are disposed;
a second printed circuit board, positioned between the first printed circuit board and the cover;
a flexible printed circuit board connecting the second printed circuit board to the first printed circuit board; and
an optical sensor disposed on the second printed circuit board and facing towards the cover.

7. The wearable device of claim 6,
wherein the second printed circuit board comprises a first connection portion electrically connected to the first electrode and a second connection portion electrically connected to the second electrode.

8. The wearable device of claim 7, further comprising:
a first conductive adhesive member electrically connecting the first connection portion and the first electrode; and
a second conductive adhesive member electrically connecting the second connection portion and the first electrode.

9. The wearable device of claim 6, wherein
the first circuit is at least partly provided on any of the first printed circuit board and the second printed circuit board,
the second circuit is at least partly provided on any of the first printed circuit board and the second printed circuit board,
the third circuit is at least partly provided on any of the first printed circuit board and the second printed circuit board, and
the fourth circuit is at least partly provided on any of the first printed circuit board and the second printed circuit board.

10. The wearable device of claim 6, further comprising a sensor circuit configured to process the first electrical signal and the second electrical signal, and the sensor circuit disposed on the second printed circuit board.

11. The wearable device of claim 6, wherein the processor comprises at least one sensor circuit configured to process the first electrical signal and the second electrical signal.

12. The wearable device of claim 6, further comprising a conductive member positioned between the first printed circuit board and the second printed circuit board, and
wherein the conductive member is electrically connected to the switch circuit and the ground of the antenna.

13. The wearable device of claim 12, wherein the switch circuit comprises:
a first pin to which the first electrode is connected;
a second pin to which the conductive member is connected; and
a third pin connected to the ground of the antenna, and
wherein the switch circuit is configured to selectively connect the first pin to at least one of the second pin and the third pin.

14. The wearable device of claim 13,
wherein the switch circuit comprises a fourth pin connected to the ground of the antenna through at least one lumped element, and
wherein the switch circuit is configured to selectively connect the first pin to at least one of the second pin, the third pin, and the fourth pin.

15. The wearable device of claim 14,
wherein the third pin is connected to the ground of the antenna through at least one other lumped element.

16. The wearable device of claims 1, wherein
the first circuit comprises any of a resistor and an inductor, and
the second circuit comprises a capacitor.

17. The wearable device of claim 1, wherein the biometric information comprises an electrocardiogram.

18. An electronic watch comprising:
a housing comprising a first cover, a second cover, and a metal frame disposed between the first cover and the second cover, the metal frame is at least partly an antenna configured to any of receive and transmit a radio frequency (RF) signal, and the second cover comprises a first electrode and a second electrode that are configured to detect an electrical signal;
at least one first circuit configured to selectively pass the electrical signal;
at least one second circuit configured to selectively pass the RF signal;
a sensor circuit coupled to the first electrode and the second electrode through the at least one first circuit and configured to process the electrical signal;
a switch circuit coupled to the first electrode and the second electrode through the at least one second circuit, and connected to a ground of the antenna; and
at least one processor operatively connected to the metal frame, the sensor circuit, and the switch circuit, and the at least one processor is configured to:
any of transmit and receive the RF signal from the antenna,
obtain biometric information, of a user, based on the electrical signal provided from the sensor circuit; and
control the switch circuit to selectively connect at least one of the first electrode and the second electrode to the ground of the antenna.

19. The electronic watch of claim 18, further comprising a conductive member disposed within the housing and electrically connected to the switch circuit, and
wherein the at least one processor is further configured to control the switch circuit to selectively connect the at least one of the first electrode and the second electrode to the conductive member.

20. The electronic watch of claim 19, wherein
the at least one first circuit comprises a first element and a second element,
the at least one second circuit comprises a third element and a fourth element,
the first element and the third element are connected in parallel to the first electrode, and
the second element and the fourth element are connected in parallel to the second electrode.
